(19) United States
(12) Patent Application Publication
(10) Pub. No.: US 2018/0098741 A1
(43) Pub. Date: Apr. 12, 2018

(54) ELECTRONIC DEVICE

(71) Applicant: Hangzhou Mega Inno of Health Technology Co. Ltd, Hangzhou (CN)

(72) Inventors: ZHAN-SHENG LU, Shenzhen (CN); CHIEH-YU LEE, New Taipei (TW)

(21) Appl. No.: 15/719,702

(22) Filed: Sep. 29, 2017

(30) Foreign Application Priority Data
Oct. 12, 2016 (CN) 201610890032.2

(51) Int. Cl. A61B 5/00; A61B 5/021; A61B 5/0428; A61B 5/1455; A61B 90/00

(52) U.S. Cl. CPC A61B 5/7475; A61B 5/02141; A61B 5/0428; A61B 5/14552; A61B 2562/227; A61B 5/7445; A61B 90/361; A61B 2560/045; A61B 2560/0493; A61B 5/6826

(57) ABSTRACT

An electronic device includes a medical testing unit. The medical testing unit includes a medical processor, a number of medical testing modules, and a number of testing subunits. Each testing subunit is electrically coupled to a corresponding medical testing module, the medical processor is electrically coupled to the medical testing modules. The medical processor is used for controlling the testing subunits and the medical testing modules to test physiological parameters of users, and acquire, process, and analyze the physiological parameters to generate health data.

### CROSS-REFERENCE TO RELATED APPLICATIONS

This application claims priority to Chinese Patent Application No. 201610890032.2 filed on Oct. 12, 2016, the contents of which are incorporated by reference herein.

### FIELD

The subject matter herein generally relates to medical devices, and particularly to an electronic device with a medical testing function.

### BACKGROUND

Medical products, such as sphygmomanometers, blood glucose meters, and ECG testers are in use. However, these medical products only have single medical testing function, which is inconvenient for users to test a number of medical items at the same time.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic view illustrating an exemplary embodiment of an electronic device.

FIG. 2 is an exploded view illustrating an exemplary embodiment of the electronic device in FIG. 1.

FIG. 3 is an exploded view from another angle of the electronic device in FIG. 2.

FIG. 4 is a block diagram illustrating an exemplary embodiment of the electronic device in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein can be practiced without these specific details. In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts have been exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” exemplary embodiment in this disclosure are not necessarily to the same exemplary embodiment, and such references mean “at least one.”

The term “comprising” means “including, but not necessarily limited to”, it specifically indicates open-ended inclusion or membership in a so-described combination, group, series, and the like.

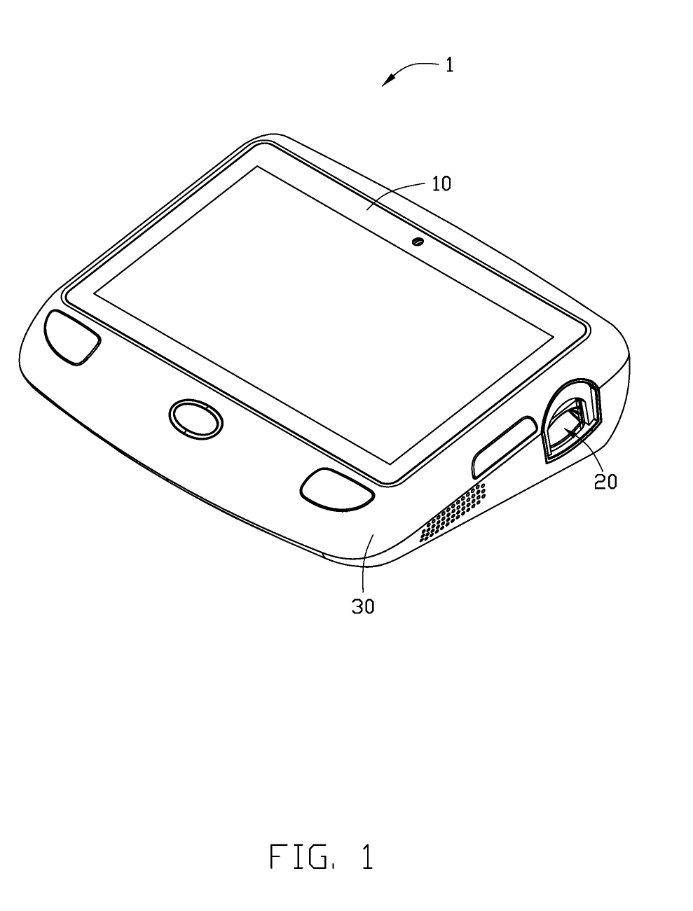

FIG. 1 illustrates an exemplary embodiment of an electronic device 1. The electronic device 1 includes, but is not limited to, a main unit 10, a medical testing unit 20, and a casing 30. In at least one exemplary embodiment, the electronic device 1 can be a smart phone, a personal computer, or a PDA (Personal Digital Assistant). FIG. 1 illustrates only one example of the electronic device 1, other examples can include more or fewer components than illustrated, or have a different configuration of the various components in other exemplary embodiments.

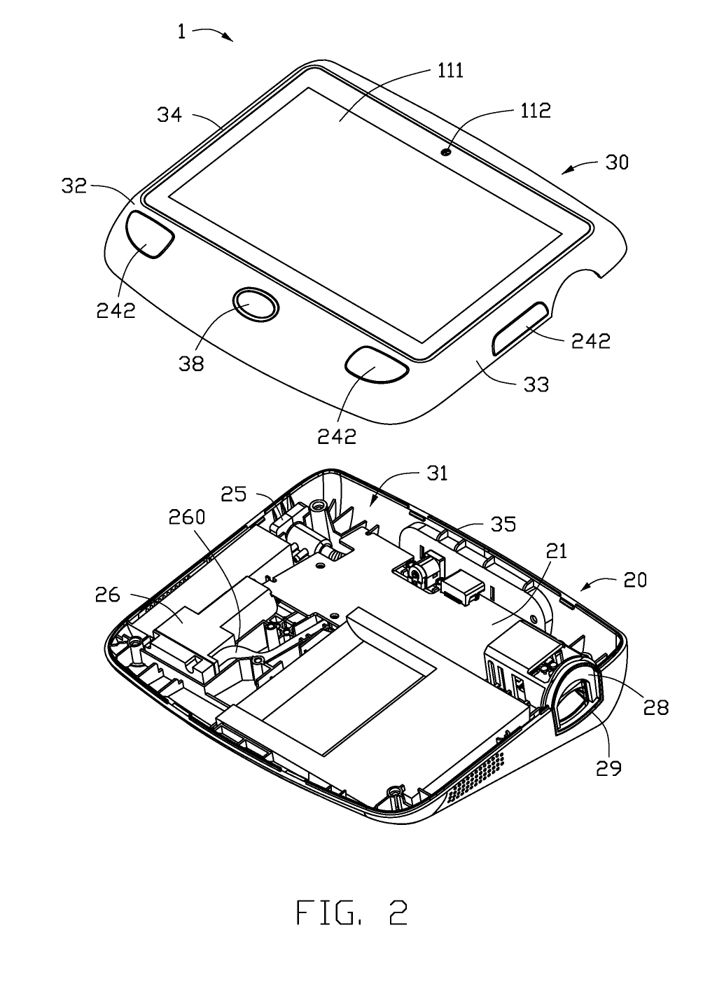

Referring to FIG. 2, the casing 30 defines a receiving space 31, and includes an upper surface 32, a first side 33, a second side 34, a third side 35, a bottom surface 36, and a bracket 37. A button 38 is arranged on the upper surface 32, the button 38 can be triggered to start or shut down the electronic device 1, and to activate or to pause a medical testing. For example, when the button 38 is pressed for longer than a first predetermined period (eg. eight seconds), the electronic device 1 starts or shuts down, when the button 38 is pressed for less than a second predetermined period (eg. two seconds), the electronic device 1 activates or pauses the medical testing.

The third side 35 is adjacent to the first side 33 and the second side 34. The bracket 37 is rotatably arranged on the bottom surface 36, the bracket 37 is used for supporting the casing 30, thus an angle is formed between the casing 30 and a supporting object.

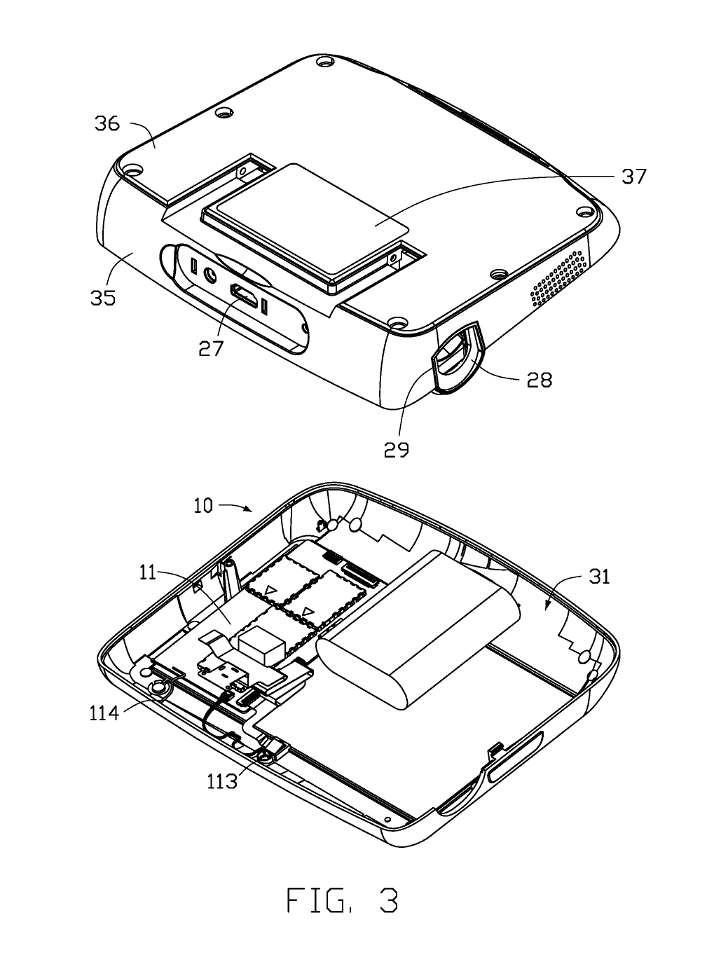

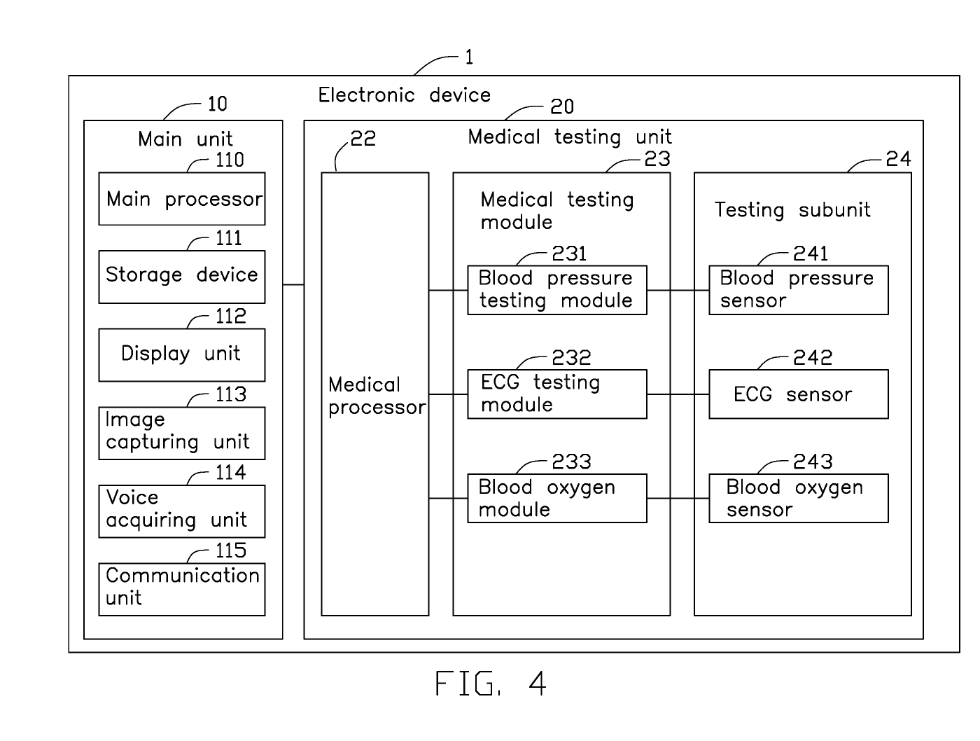

Referring to FIG. 3, the main unit 10 and the medical testing unit 20 are received in the receiving space 31 of the casing 30. The main unit 10 is used for performing basic functions of the electronic device 1, such as web surfing, video and audio playing, mobile communication, and wireless communication etc. In at least one exemplary embodiment, the main unit 10 at least includes a main board 11. A main processor 110 (shown in FIG. 4) is arranged on the main board 11. In at least one exemplary embodiment, the main processor 110 can be a central processing unit (CPU), a microprocessor, or other data processor chip that performs functions of the electronic device 1, such as SoC (System on Chip).

Referring to FIG. 4, the main unit 10 further includes a storage device 111, a display unit 112, an image capturing unit 113, a voice acquiring unit 114, and a communication unit 115. In at least one exemplary embodiment, the storage device 111 can include various types of non-transitory computer-readable storage mediums. For example, the storage device 111 can be an internal storage system, such as a flash memory, a random access memory (RAM) for temporary storage of information, and/or a read-only memory (ROM) for permanent storage of information. The storage device 111 can also be an external storage system, such as a hard disk, a storage card, or a data storage medium.

In at least one exemplary embodiment, the display unit 112 can be a touch screen. The display unit 112 is arranged in the casing 30, and is used for displaying media content of the electronic device 1 and health data of users. The image capturing unit 113 can be a camera, the image capturing unit 113 is arranged in the casing and exposed out of the display unit 112. The image capturing unit 113 is used for capturing images and recording videos. The voice acquiring unit 114 can be a microphone, the voice acquiring unit 114 is used for acquiring voice of users. The communication unit 115 includes a wireless communication module, the wireless communication module is used for performing WI-FI communication, BLUETOOTH communication, 2G communication, 3G communication, and/or 4G communication.

The medical texting unit 20 at least includes a medical board 21. The medical board 21 is integrated with a medical processor 22, a number of medical testing modules 23, and a number of testing subunits 24. Each testing subunit 24 corresponds to a medical testing module 23. In at least one exemplary embodiment, the medical processor 22 can be a MCU (Microcontroller Unit). The medical board 21 is electrically coupled to the main board 11. Each testing subunit 24 is electrically coupled to a medical testing module 23. The medical processor 22 is used for controlling the testing subunits 24 and the medical testing modules 23 to test physiological parameters of the users, and acquire, process, and analyze the physiological parameters to generate the health data.

The medical testing unit 20 is electrically coupled to the main processor 110. The medical testing unit 20 can transmit the generated health data to the main processor 110. A health management application (not shown) is run in the electronic device 1, thus the main processor 110 can process (eg. store, display, or transmit) the health data in combination with the health management application.

In at least one exemplary embodiment, the electronic device 1 makes the best use of space by defining two main boards (the main board 11 and the medical board 21), thus avoiding tight gaps between the electronic components of the electronic device 1. In addition, the main processor 110 and the medical processor 22 share out the work and cooperate with each other, to avoid single processor performing too many functions and being overloaded. In other exemplary embodiment, the electronic device 1 also can include one main board. The main unit 10 and the medical testing unit 20 are arranged on the main board.

In at least one exemplary embodiment, the medical testing modules 23 include, but are not limited to, a blood pressure testing module 231, an ECG (electrocardiogram) testing module 232, and a blood oxygen testing module 233. The modules 231-233 can be collections of software instructions stored in the electronic device 1 and executed by the medical processor 22. The modules 231-233 also can include functionality represented as hardware or integrated circuits, or as software and hardware combinations, such as a special-purpose processor or a general-purpose processor with special-purpose firmware.

The testing subunits 24 includes, but are not limited to, a blood pressure sensor 241, at least three ECG sensors 242, and a blood oxygen sensor 243.

As illustrated in FIG. 2, in the least one exemplary embodiment, a blood testing port 25 and an air pump 26 are arranged on the medical board 21. The blood testing port 25 is exposed out of the second side 34 of the casing 30. The blood pressure sensor 241 and the air pump 26 are connected to a blood pressure testing sleeve (not shown) through the blood pressure testing port 25. The blood pressure sensor 241 is used for detecting pressure signals. The air pump 26 is used for inflating or deflating the blood pressure testing sleeve.

When testing the blood pressure parameter of a user, one end of the blood pressure testing sleeve is connected to the blood pressure testing port 25, and the other end is sleeved on an arm of the user. The medical processor 22 controls the air pump 26 to inflate the blood pressure testing sleeve, thus applying pressure to the arm of the user, and can control the air pump 26 to deflate the blood pressure testing sleeve, thus removing the applied pressure. The blood pressure sensor 241 detects the pressure signals during inflating or deflating the blood pressure testing sleeve, and transmits the pressure signals to the blood pressure testing module 231, then the blood pressure testing module 231 can determine the blood pressure parameter of the user according to the pressure signals.

In at least one exemplary embodiment, the testing subunits 24 include four ECG sensors 242. Two ECG sensors 242 are arranged on the upper surface 32 of the casing 30, the other two ECG sensors 242 are respectively arranged on the first side 33 and the second side 34 of the casing 30. The ECG sensor 242 can be an ECG signal acquiring electrode.

When testing the ECG of the user, the user can touch any three ECG sensors 242 with three fingers, then the ECG sensors 242 can detect the ECG signals of the user, and transmit the ECG signals to the ECG testing module 232. The ECG testing module 232 can determine the ECG of the user according to the ECG signals.

In at least one exemplary embodiment, the medical board 21 further defines an ECG testing port 27, the ECG testing port 27 is exposed out of the third side 34 of the casing 30, and is used for connecting with an external ECG testing device (not shown).

In at least one exemplary embodiment, the blood oxygen sensor 243 can be an infrared sensor. The blood oxygen sensor 243 is used for receiving and transmitting infrared rays. The main board 21 further includes a finger clamping device 28 and a blood oxygen testing port 29, the finger clamping device 28 and the blood oxygen testing port 29 are arranged on the first side 33 of the casing 30. The finger clamping device 28 is exposed out of the blood oxygen testing port 29, and is used for clamping at least one finger of the user.

When testing the blood oxygen parameter of the user, the user can insert the finger into the finger clamping device 28. The blood oxygen sensor 243 transmits infrared rays, receives the infrared rays reflected by the user finger, and feeds the transmitted infrared signals and received infrared signals back to the blood oxygen testing module 233. The blood oxygen testing module 233 can determine the blood oxygen parameter of the user according to the infrared signals.

In at least one exemplary embodiment, the electronic device 1 further can be connected to other external medical devices through the communication unit 115, such as blood glucose testing devices, body fat testing devices etc., and acquire the relevant physiological readings, such as blood glucose and body fat levels or values, from these medical devices.

In at least one exemplary embodiment, the electronic device 1 further can adopt the communication unit 115 for network communication, such as the Internet communication, the 2G communication, the 3G communication, or the 4G communication. Thus, the user can make a video call or voice call with online doctors, and transmit the health data to the online doctors, then the online doctors can evaluate a health index of the user according to the health data, and issue advice.

In at least one exemplary embodiment, not only does the electronic device 1 have basic computer functions, but also has medical testing functions, and can connect to the online doctors through the network communication, which is convenient for the users.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being exemplary embodiments of the present disclosure.

What is claimed is:

1. An electronic device comprising:
a medical testing unit comprising a medical processor, a plurality of medical testing modules, and a plurality of testing subunits, wherein each testing subunit is electrically coupled to a corresponding medical testing module, the medical processor is electrically coupled to the medical testing modules, the medical processor is used for controlling the testing subunits and the medical testing modules to test physiological parameters of users, and acquire, process, and analyze the physiological parameters to generate health data.

2. The electronic device according to claim 1, further comprising:
a main unit comprising a main processor, wherein the main processor is electrically coupled to the medical testing unit, the main processor is used for receiving and processing the generated health data.

3. The electronic device according to claim 2, wherein the main unit further comprising a main board, the main processor is arranged on the main board; the medical testing unit further comprising a medical board, the medical processor, the plurality of medical testing modules, and the plurality of testing subunits are arranged on the medical board.

4. The electronic device according to claim 2, further comprising:
a casing defining a receiving space, wherein the receiving space is used for receiving the main unit and the medical testing unit.

5. The electronic device according to claim 4, further comprising:
a bracket rotatably mounted on the casing, wherein the bracket is used for supporting the casing.

6. The electronic device according to claim 4, further comprising:
a button mounted on the casing, wherein the button is used for starting or shutting down the electronic device, and activating or pausing a medical testing.

7. The electronic device according to claim 2, wherein the main unit further comprising:
a storage device; and/or
a display unit used for displaying media content of the electronic device and the health data of the users; and/or
an image capturing unit used for capturing images and recording videos; and/or
a voice acquiring unit used for acquiring voice of the users.

8. The electronic device according to claim 2, wherein the main unit further comprising:
a communication unit used for performing WiFi communication, bluetooth communication, 2G communication, 3G communication, and/or 4G communication, wherein the electronic device acquires the health data from external medical devices through the communication unit.

9. The electronic device according to claim 1, wherein the plurality of medical testing modules comprising a blood pressure testing module, the plurality of testing subunits comprising a blood pressure sensor, wherein the blood pressure testing module and the blood pressure sensor are used for testing blood pressure parameter of the user.

10. The electronic device according to claim 9, further comprising a blood testing port and an air pump, wherein the blood pressure sensor and the air pump are connected to a blood pressure testing sleeve through the blood pressure testing port, the blood pressure sensor is used for detecting pressure signals, the air pump is used for inflating or deflating the blood pressure testing sleeve.

11. The electronic device according to claim 1, wherein the plurality of medical testing modules comprising an ECG testing module, the plurality of testing subunits comprising at least three ECG sensors, wherein the ECG testing module and the ECG sensors are used for testing ECG parameter of the user.

12. The electronic device according to claim 11, wherein the electronic device further defines an ECG testing port, the ECG testing port is used for connecting with an external ECG testing device.

13. The electronic device according to claim 1, wherein the plurality of medical testing modules comprising a blood oxygen testing module, the plurality of testing subunits comprising a blood oxygen sensor, wherein the blood oxygen testing module and the blood oxygen sensor are used for testing blood oxygen parameter of the user.

14. The electronic device according to claim 13, wherein the blood oxygen sensor is an infrared sensor, the blood oxygen sensor is used for receiving and transmitting infrared rays, the electronic device further comprising a finger clamping device, the finger clamping device is used for clamping at least one finger of the user.
